(19) United States
(12) Patent Application Publication
(10) Pub. No.: US 2009/0297603 A1
(43) Pub. Date: Dec. 3, 2009

(54) INTERSPINOUS DYNAMIC STABILIZATION SYSTEM WITH ANISOTROPIC HYDROGELS

(76) Inventor: Abhijeet JOSHI, Austin, TX (US)

Correspondence Address: SPRINKLE IP LAW GROUP, 1301 W. 25TH STREET, SUITE 408, AUSTIN, TX, 78705, US

(21) Appl. No.: 12/128,766

(22) Filed: May 29, 2008

(51) Int. Cl. A61K 9/00; A61F 2/44

(52) U.S. Cl. 424/484; 623/17.12

(57) ABSTRACT

Disclosed are embodiments of an interspinous dynamic stabilization system that can uniquely address the dynamic stabilization of a spinal segment and facet joint concurrently and that can be useful for drug delivery applications. The interspinous dynamic stabilization system comprises a relatively rigid casing and relies on the anisotropic expansion feature of specially manufactured hydrogels contained in the casing to resist and control the extension of the spine. In a surgical procedure, the casing is attached to adjacent spinous processes laterally or in an anterior-posterior direction. Dehydrated hydrogels are then inserted inside the casing, perhaps one by one in a minimally invasive manner. Upon absorption of fluid, the hydrogels swell axially in the preferred direction, eventually lifting the superior spinous process. As the casing is attached to the spinous processes, the interspinous dynamic stabilization system can also have enhanced stability against torsional and lateral bending.

### TECHNICAL FIELD OF THE DISCLOSURE

This disclosure relates generally to spinal implants. More particularly, the present disclosure relates to an interspinous dynamic stabilization system which can uniquely address the dynamic stabilization of a spinal segment and facet joint concurrently and which can be useful as a drug delivery device. The present disclosure also relates to methods of implanting such an interspinous dynamic stabilization system in a patient.

### BACKGROUND OF THE RELATED ART

The human spine consists of segments known as vertebrae linked by intervertebral disks and held together by ligaments. There are 24 movable vertebrae—7 cervical (neck) vertebrae, 12 thoracic (chest) vertebrae, and 5 lumbar (back) vertebrae. Each vertebra has a somewhat cylindrical bony body (centrum), a number of winglike projections (processes), and a bony arch. The arches are positioned so that the space they enclose forms the vertebral canal. The vertebral canal houses and protects the spinal cord, and within it the spinal fluid circulates. Ligaments and muscles are attached to various projections of the vertebrae. The bodies of the vertebrae form the supporting column of the skeleton. Five fused vertebra make up the sacrum and coccyx, the very bottom of the vertebral column.

The spine is subject to abnormal curvature, injury, infections, tumor formation, arthritic disorders, and puncture or slippage of the cartilage disks. Injury or illness, such as spinal stenosis and prolapsed discs may result in intervertebral discs having a reduced disc height, which may lead to pain, loss of functionality, reduced range of motion, and the like. Scoliosis is one relatively common disease which affects the spinal column. It involves moderate to severe lateral curvature of the spine, and, if not treated, may lead to serious deformities later in life. One treatment involves surgically implanting devices to correct the curvature.

In addition to spinal stenosis, other conditions such as spinal arthritis, facet joint disease, sprains and strains, soft tissue diseases, and acute disc herniations tend to be worsened by extension of the spine (bending backward) and relieved by flexion (bending forward) or the neutral position. For example, the facet joint is loaded or compressed on extension and unloaded and stretched on flexion. They have been found to be a source of pain in patients presenting with low back pain and can refer pain into the lower extremity. In the case of thoracic extension dysfunctions, which may include rotation and lateral bending dysfunctional elements, compensation for such extension restrictions may occur in the lower lumbar spine, in the form of increased extension. Increased extension can increase pressure on, the spinal cord and cause increased posterior disc and facet compression. The same principle applies to upper lumbar extension restrictions. Increased extension can thus lead to low back pain, hip pain, and even knee complaints. A non-surgical treatment may be a physical therapy program directed at minimizing stress to the painful area while improving the biomechanics by stretching structures that have become tight and strengthening the muscles that support and unload these painful areas. In some cases, anesthetic injections can be used to confirm the source of pain and perhaps control the symptoms.

Modern spine surgery often involves spinal fixation through the use of spinal implants or fixation systems to correct or treat various spine disorders or to support the spine. Spinal implants may help, for example, to stabilize the spine, correct deformities of the spine, facilitate fusion, or treat spinal fractures.

A spinal fixation system typically includes corrective spinal instrumentation that is attached to selected vertebra of the spine by screws, hooks, and clamps. The corrective spinal instrumentation includes spinal rods or plates that are generally parallel to the patient's back. The corrective spinal instrumentation may also include transverse connecting rods that extend between neighboring spinal rods. Spinal fixation systems are used to correct problems in the cervical, thoracic, and lumbar portions of the spine, and are often installed posterior to the spine on opposite sides of the spinous process and adjacent to the transverse process.

Often, spinal fixation may include fused and/or rigid support for the affected regions of the spine. Such systems when implanted inhibit movement in the affected regions in virtually all directions. More recently, so called “dynamic” systems have been introduced. These systems allow at least some movement (e.g., flexion, extension, lateral bending, or torsional rotation) of the affected regions of the spine in at least some of the directions.

### SUMMARY OF THE DISCLOSURE

Embodiments of an interspinous dynamic stabilization system disclosed herein take advantage of existing technologies to uniquely and simultaneously provide dynamic stabilization of a spinal segment and facet joint in a minimally invasive manner. Embodiments of the interspinous dynamic stabilization system disclosed herein rely on the anisotropic expansion feature of specially manufactured hydrogels to resist and control the extension of the spine.

In some embodiments, an interspinous dynamic stabilization system may comprise a hydrogel manufactured to expand axially in a predetermined direction upon absorption of fluid and a casing for constraining or housing the hydrogel. In some embodiments, the casing may comprise a top surface conforming to a bottom portion of a superior spinous process and a bottom surface conforming to a top portion of an inferior spinous process. Upon absorption, the hydrogel in hydrated form can lift the superior spinous process, advantageously providing dynamic spinal stabilization and relieving facet joint pain.

According to embodiments disclosed herein, the casing may vary from implementation to implementation. In some embodiments, the casing has folds for accommodating expansion of the hydrogel. In some embodiments, the casing is partially enclosed. In some embodiments, the casing comprises tabs for attaching to the superior spinous process and to the inferior spinous process. In some embodiments, the attachment may be bi-lateral or in an anterior-posterior direction. In some embodiments, the casing may comprise a pocket area where the hydrogel is to be constrained or housed. In some embodiments, one of the tabs may extend over the pocket area, leaving a gap through which the hydrogel can be inserted.

In some embodiments, the casing may have its own dampening elements. In some embodiments, the casing comprises an upper portion, a lower portion, and dampening elements. In some embodiments, the top surface of the casing is part of the upper portion of the casing, the bottom surface of the casing is part of the lower portion of the casing, and the dampening elements are positioned between the upper portion and the lower portion of the casing.

In some embodiments, the casing may comprise side walls, each of which may have two or more holes. Bone fasteners may be utilized to attach these side walls bi-laterally to adjacent spinous processes through those holes, leaving a space between the adjacent spinous processes and the side walls where the hydrogel may be constrained and directly attachable to the adjacent spinous processes.

According to embodiments disclosed herein, the casing may be made of any suitable biocompatible materials, including metal and composite, and the hydrogel is specially manufactured to expand axially in a predetermined direction upon absorption of fluid. In one embodiment, the hydrogel is radially compressed to a bullet form, making it particularly suitable for minimally invasive easy insertion. In some embodiments, the exterior or interacting surface of the hydrogel is made bioactive, making these embodiments particularly suitable for drug delivery applications.

Embodiments disclosed herein include methods of implanting an interspinous dynamic stabilization system. One embodiment may comprise the steps of making an incision in a patient, placing a casing of the interspinous dynamic stabilization system between adjacent spinous processes of a spinal segment of the patient, and inserting one or more units of a hydrogel in dehydrated form into the casing through an opening thereof. Upon absorption of fluid, the hydrogel expands axially in a predetermined direction, lifting the superior spinous process. The method may further comprise supplying a saline solution to the hydrogel to speed up the swelling process. In some embodiments, the method may further comprise preparing the bottom portion of the superior spinous process and the top portion of the inferior-spinous process to accommodate the top surface and the bottom surface of the casing. In embodiments where the hydrogel has a bioactive surface, the method may further comprise utilizing the interspinous dynamic stabilization system as a drug delivery device.

In some embodiments, a method of implanting an interspinous dynamic stabilization system may comprise the steps of making an incision in a patient, attaching, bi-laterally or in an anterior-posterior direction, a casing of the interspinous dynamic stabilization system to adjacent spinous processes of a spinal segment of the patient, and inserting one or more units of a hydrogel in dehydrated form into the casing through an opening thereof. The method may further comprise hydrating the hydrogel by supplying a saline solution to the hydrogel. In some embodiments of an interspinous dynamic stabilization system, the casing may have an open-style that allows the hydrogel constrained therein to directly attach to both the superior spinous process and the inferior spinous process when the hydrogel is fully hydrated. The hydrogel thus utilized may have a bioactive exterior or interacting surface, making the interspinous dynamic stabilization system particularly useful for drug delivery purposes.

Embodiments of the interspinous dynamic stabilization system disclosed herein can provide many advantages, including but not limited to, reducing, resisting, and controlling the extension of the spine in order to achieve the following: soft (dynamic) stabilization of the affected spinal segment; minimize the loads experienced by facet joints in a damaged disc/spine; minimize the facet joint articulation for pain relief; drug delivery device/carrier; and minimally invasive surgery and faster recovery.

Other objects and advantages of the embodiments disclosed herein will be better appreciated and understood when considered in conjunction with the following description and the accompanying drawings.

### BRIEF DESCRIPTION OF THE DRAWINGS

A more complete understanding of the present invention and the advantages thereof may be acquired by referring to the following description, taken in conjunction with the accompanying drawings in which like reference numbers indicate like features and wherein:

FIG. 1(A) depicts a spinal segment in a forward bending (flexion) position, showing a facet joint being unloaded and stretched;

FIG. 1(B) depicts the same spinal segment in a backward stretching (extension) position, showing the facet joint being loaded or compressed;

FIG. 2 depicts a portion of the spine having vertebral bodies separated by discs;

FIG. 3 depicts one embodiment of an interspinous dynamic stabilization system that can address the dynamic stabilization of the spinal segment and the facet joint concurrently;

FIG. 4 depicts one embodiment of an interspinous dynamic stabilization system implanted between adjacent spinous processes;

FIG. 5 depicts one embodiment of an interspinous dynamic stabilization system comprising a casing and specially manufactured anisotropic hydrogels;

FIG. 6(A) depicts a top view of a hydrogel in dehydrated form;

FIG. 6(B) depicts a side view of the hydrogel in dehydrated form;

FIG. 6(C) depicts a perspective view of the hydrogel in dehydrated form;

FIG. 6(D) depicts a side view of the hydrogel in hydrated form;

FIG. 7 depicts one embodiment of an interspinous dynamic stabilization system comprising a casing with accordion-like features;

FIG. 8 depicts one embodiment of an interspinous dynamic stabilization system comprising a casing with tabs for attaching the casing to adjacent spinous processes in the anterior-posterior direction;

FIG. 9 depicts one embodiment of an interspinous dynamic stabilization system comprising a casing with tabs for laterally attaching the casing to adjacent spinous processes;

FIG. 10 depicts one embodiment of an interspinous dynamic stabilization system comprising a casing with side walls and open on the top and bottom;

FIG. 11 depicts an example of a casing wall; and

FIG. 12 depicts one embodiment of an interspinous dynamic stabilization system comprising a casing with dampening elements.

While this disclosure is susceptible to various modifications and alternative forms, specific embodiments thereof are shown by way of example in the drawings and will herein be described in detail. It should be understood, however, that the drawings and detailed description thereto are not intended to limit the disclosure to the particular form disclosed, but to the contrary, the intention is to cover all modifications, equivalents and alternatives falling within the spirit and scope of the present disclosure as defined by the appended claims.

### DETAILED DESCRIPTION

The inventive interspinous dynamic stabilization system and the various features and advantageous details thereof are explained more fully with reference to the non-limiting embodiments detailed in the following description. Descriptions of well known starting materials, manufacturing techniques, components and equipment are omitted so as not to unnecessarily obscure the invention in detail. Skilled artisans should understand, however, that the detailed description and the specific examples, while disclosing preferred embodiments of the invention, are given by way of illustration only and not by way of limitation. Various substitutions, modifications, and additions within the scope of the underlying inventive concept(s) will become apparent to those skilled in the art after reading this disclosure. Skilled artisans can also appreciate that the drawings disclosed herein are not necessarily drawn to scale.

As used herein, the terms “comprises,” “comprising,” includes, “including,” “has,” “having” or any other variation thereof, are intended to cover a non-exclusive inclusion. For example, a process, product, article, or apparatus that comprises a list of elements is not necessarily limited to only those elements, but may include other elements not expressly listed or inherent to such process, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Additionally, any examples or illustrations given herein are not to be regarded in any way as restrictions on, limits to, or express definitions of, any term or terms with which they are utilized. Instead these examples or illustrations are to be regarded as being described with respect to one particular embodiment and as illustrative only. Those of ordinary skill in the art will appreciate that any term or terms with which these examples or illustrations are utilized encompass other embodiments as well as implementations and adaptations thereof which may or may not be given therewith or elsewhere in the specification and all such embodiments: are intended to be included within the scope of that term or terms. Language designating such non-limiting examples and illustrations includes, but is not limited to: “for example,” “for instance,” “e.g.,” “in one embodiment,” and the like.

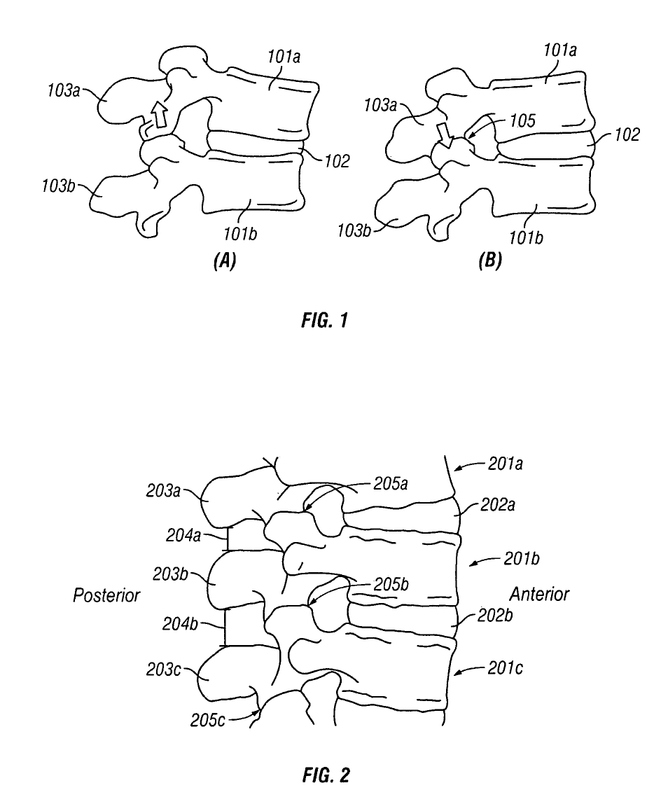

FIG. 1(A) depicts a spinal segment in a forward bending (flexion) position, showing facet joint 105 being unloaded and stretched. FIG. 1(B) depicts the same spinal segment in a backward stretching (extension) position, showing facet joint 105 being loaded or compressed. In FIG. 1(A) and 1(B), the spinal segment includes two vertebrae and an intervertebral disc. The vertebral body is the main portion of the vertebra and bears about 80% of the load while standing. At the anterior (front) part of the spinal segment, disc 102 separates vertebral bodies 101a and 101b and acts as a shock absorber for the spinal segment. Disc 102 is made up of fibrous layers called the annulus surrounding a gel-like substance called the nucleus pulposus or nucleus. At the posterior part of the spinal segment, the vertebral arch is formed by a pair of pedicles, a pair of laminae, and supports seven processes—four articular, two transverse, and one spinous. As FIG. 1(A) and FIG. 1(B) illustrate, adjacent spinous process 103a and spinous process 103b are pulled away from one another in flexion and pushed towards each other in extension. The range of movement is restricted by superior and inferior articular facets forming facet joint 105.

FIG. 2 depicts a portion of the spine having vertebral bodies 201a, 201b, 201c separated by discs 202a and 202b. In the example of FIG. 2, space 204a between adjacent spinous process 203a and spinous process 203b is less than space 204b between adjacent spinous process 203b and spinous process 203c. This may be caused by one or more factors, such as disease, injury, or age. For example, disc 202a may suffer from disc degeneration disease (DDD), where the jelly-like substance of the intervertebral disc becomes dry and stiff, losing its cushioning effect and no longer can work as a shock absorber. DDD is attributed to the degenerative process in the spine and is a common cause for chronic or recurring back pain. Patients with DDD may have back pain, leg pain, or varying degrees of both. DDD generally leads to loss of disc height and alters the normal spinal biomechanics and motion. In this case, loss of disc height can reduce the separation of facet joints 205a, 205b, or even 205c and alter the biomechanics of those joints. The cartilage of the joint may become compromised or destroyed resulting in nerve compression and/or bone-on-bone contact in the joint. Structural instability and nerve compression are causes for persistent and often significant pain. Furthermore, the abnormal movement of the degenerative disc or motion segment forces facet joints 205a, 205b, or even 205c to carry abnormal physiologic loads that, in turn, cause facet degeneration.

In some cases, surgery may be required to prevent the spine from pressing on the spinal cord and/or to stabilize the affected vertebrae. One treatment option involves preventing the spine from overextension while restoring a natural height of the space between adjacent spinous processes when the spine is in the neutral position.

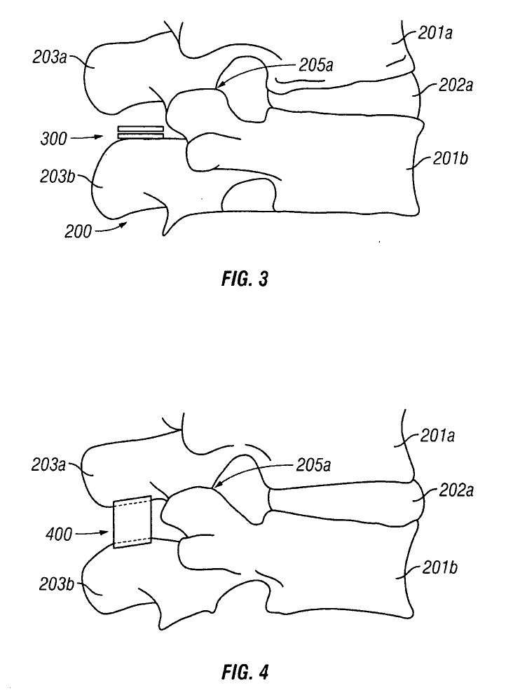

FIG. 3 depicts one embodiment of interspinous dynamic stabilization system 300 that can be placed between adjacent spinous process 203a and spinous process 203b to address the dynamic stabilization of spinal segment 200 and facet joint 205a concurrently. Embodiments of an interspinous dynamic stabilization system disclosed herein rely on the anisotropic expansion feature of specially manufactured hydrogels to reduce, resist and control the extension of the spine. This allows the interspinous dynamic stabilization system to achieve the following objectives, in some cases simultaneously:
soft (dynamic) stabilization of the affected spinal segment;
minimize the loads experienced by facet joints in a damaged disc/spine;
minimize the facet joint articulation for pain relief;
act as a drug delivery device/carrier; and
minimally invasive surgery and faster recovery.

Hydrogels, in general, are hydrophilic (water loving) in nature. They can absorb water, body fluid, etc. and can expand up to 200 to 400% of their initial volume. This expansion is generally isotropic, which means that they will swell in equal amount in all directions. Using special manufacturing techniques, such as those disclosed by U.S. Pat. No. 7,204,897, and U.S. Patent Application Publication No. 2005/0171611, both of which are incorporated herein by reference, the hydrogel expansion can be made anisotropic, which means that the specially manufactured hydrogel will expand only in the preferred direction (say, Z) and will not expand, or at least not significantly, in two other directions (say, X and Y). Hydrogels with anisotropic expansion have been successfully manufactured for spinal nucleus implants (e.g., NeuDisc by Replication Medical Inc. of New Jersey). These hydrogels are traditionally used for spinal nucleus implants and will expand in axial direction by absorbing water, body fluid, etc.

Using special techniques, such as those disclosed by U.S. Patent Application Publication No. 2006/0136065, hydrogels can also be made in a variety of shapes in a dry-state” or “pre-insertion state”, some of which may be suitable for minimally invasive insertion. U.S. Pat. No. 6,264,695, issued to Stoy, describes a swellable plastic that, in folded form, can be inserted, through an incision, into a cavity of a spinal disc. After insertion, the swellable plastic is then unfolded and hydrated within the cavity to replace a portion of nucleus pulposus tissue removed from the spinal disc.

As one skilled in the art can appreciate, hydrogels can be reinforced using a variety of materials, including, but not limited to, polyester fiber, polyester mesh, Dacron® mesh, etc. Dacron is a ® ™ of Invista, Inc. These hydrogels may have the ability to exert swelling force (i.e., lifting force) in the range of 100 newton (N) to 800 N, depending upon the composition and upon absorption of fluids. All these features can be advantageously used to the objectives mentioned above.

In the example of FIG. 3, the hydrogel when implanted would absorb the fluid and swell to increase the space between spinous process 203a and spinous process 203b, eventually lifting superior spinous process 203a and thereby resisting extension. In some embodiments, the use of saline is recommended for faster swelling of the hydrogel.

FIG. 4 depicts one embodiment of interspinous dynamic stabilization system 400 implanted between superior spinous process 203a and inferior spinous process 203b. According to embodiments disclosed herein, the spinous processes (superior and inferior) act as an anchor for the casing of the interspinous dynamic stabilization system. In some embodiments, the casing contains a number of dry-state (i.e., dehydrated) anisotropic hydrogels prior to implantation. Hydrogels that are suitable for implementing embodiments of an interspinous dynamic stabilization system disclosed herein may be manufactured in a variety of shapes, including, but not limited to, thin wafer, bullet, springs, coils, capsules, etc. In some embodiments, anisotropic hydrogels may be manufactured by radially compressing to have a bullet shape for minimally invasive easy insertion. In some embodiments,. anisotropic hydrogels suitable for implementing embodiments of the interspinous dynamic stabilization system disclosed herein may be implanted one unit at a time.

In practice, the desired spacing between the adjacent spinous processes will determine the number of hydrogel units required. In some embodiments, the casing of an interspinous dynamic stabilization system for treatment of a single level spinal segment may contain one or more hydrogel units.

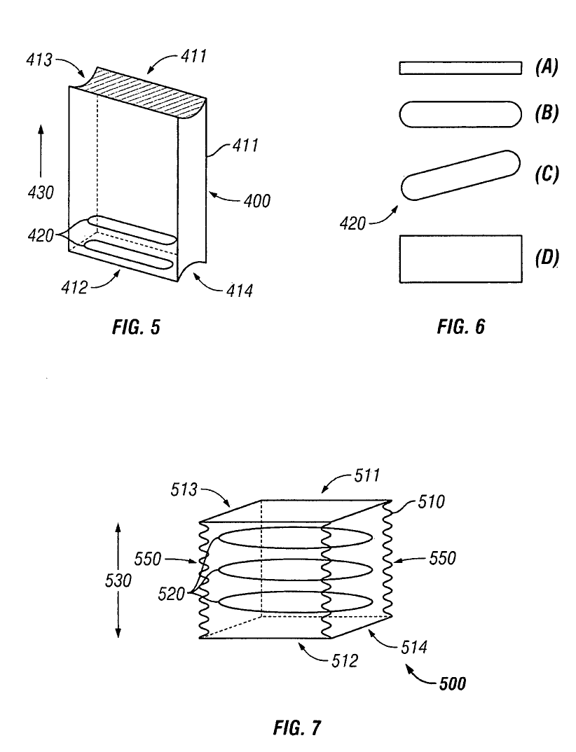

FIG. 5 depicts one embodiment of interspinous dynamic stabilization system 400 comprising casing 410 and hydrogel 420. In this example, casing 410 has a body with surfaces 411 and 412. In some embodiments, top surface 411 may have profile 413 for engaging superior spinous process 203a from the bottom of superior spinous process 203a and bottom surface 412 may have profile 414 for engaging inferior spinous process 203b from the top of inferior spinous process 203b. In some embodiments, casing 410 may be fully enclosed. In some embodiments, casing 410 may be partially enclosed. Casing 410 can be made of any biocompatible material with a structure that permits a flexion and an extension of the spinal column on either side of a neutral position of the spine.

In FIG. 5, two units of hydrogel 420 are shown in dehydrated form. FIGS. 6(A), (B), and (C) depict a top view (A), a side view (B), and a perspective view (C) of a unit of hydrogel 420 in dehydrated form. In some embodiments, a unit of hydrogel 420 in dehydrated form may have a height of about 2 mm and a length of about 15 to 25 mm. Other sizes are also possible. When hydrated, hydrogel 420 will swell in the direction as indicated by arrow 430 inside casing 410 (see FIG. 5). FIG. 6(D) depicts a side view of the unit of hydrogel 420 in hydrated form. Depending upon water absorption and other factors, the height of hydrogel 420 in hydrated form may vary by implementation.

The casing may also vary from implementation to implementation, so long as it is formed with features that can constrain and/or house the hydrogel. The shape and features of the casing should be adapted so that they are similar to the portion of the spinous processes to which the casing attaches. In some cases, preparation of spinous process(es) may be required to conform to the casing.

FIG. 7 depicts one embodiment of interspinous dynamic stabilization system 500 comprising casing 510 and hydrogel 520. In this example, casing 510 has a body with surfaces 511 and 512, and features 550. In some embodiments, top surface 511 may have profile 513 for engaging a superior spinous process from the bottom thereof and bottom surface 512 may have profile 514 for engaging an inferior spinous process from the top thereof. In some embodiments, features 550 may take the form of pleats or folds, giving casing 510 an accordion-like or spring-like ability to expand along with hydrogel 520 in either or both directions as indicated by arrows 530. Casing 510 may be fully enclosed or partially enclosed.

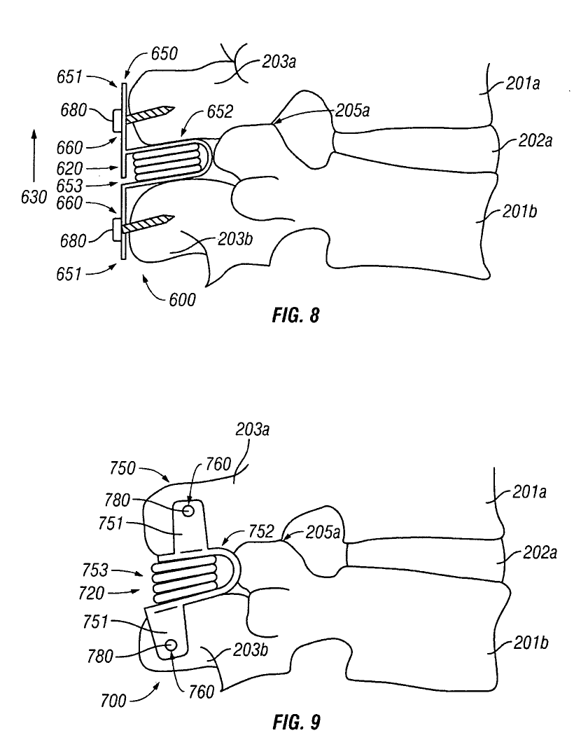

FIG. 8 depicts one embodiment of interspinous dynamic stabilization system 600 comprising casing 650 and hydrogel 620. In this example, casing 650 comprises pocket 652 for housing hydrogel 620. Pocket 652 is structured to be inserted in the space between superior spinous process 203a and inferior spinous process 203b. In some embodiments, pocket 652 may have an upper surface that conforms to the bottom portion of superior spinous process 203a and a lower surface that conforms to the top portion of inferior spinous process 203b.

Casing 650 further comprises tabs 651 for attaching casing 650 to superior spinous process 203a and inferior spinous process 203b. Tabs 651 are connected to pocket 652 on either end of pocket 652 and can be formed separate from or monolithically with pocket 652. Each tab 651 may have at least one hole 660 through which bone fastener 680 can be fastened or otherwise secured onto a spinous process. Suitable bone fasteners 680 may include, but are limited to, bone screws. In the example of FIG. 8, one of tabs 651 is extended over pocket 652, leaving casing 650 partially enclosed with gap 653.

In some embodiments, units of hydrogel are inserted into the casing prior to surgery or prior to attaching the casing to the adjacent spinous processes during a surgical procedure. In some embodiments, during a surgical procedure, once the casing is attached to the adjacent spinous processes using bone fasteners, units of hydrogel are then inserted, perhaps one by one, inside the casing between the spinous processes. As FIG. 8 illustrates, once casing 650 is attached to superior spinous process 203a and inferior spinous process 203b via bone fasteners 680, at least one unit of hydrogel 620 in dehydrated form may be inserted through slit or gap 653. Hydration of hydrogel 620 will cause hydrogel 620 to swell up in the direction as indicated by arrow 630, eventually lifting superior spinous process 203a. In some embodiments, to expedite the swelling of hydrogels and lifting of the superior supinous process, a saline solution may be injected within the casing. For example, through gap 653, additional fluid may be supplied to hydrogel 620.

Upon absorption of the fluid, the superior spinous process will experience the lifting force due to an anisotropic expansion of the hydrogels and the distance between the adjacent spinous processes will be increased. This process will occur within the first 4 to 18 hours. This lifting can minimize the loads experienced by facet joints and can also minimize the painful articulation between the interacting fact joints.

In some embodiments, the casing can be monolithically made of a metal material. In some embodiments, the metal material is titanium. The relatively rigid casing can provide the stability to the affected spinal segment and the hydrogel material within the casing can act as a “cushion” or “dampening element,” providing a unique blend of stability and range of motion (ROM) in the flexion-extension direction. In some embodiments, the casing can be monolithically made of a composite material to induce additional ROM without affecting stability.

Depending upon the casing design, in some embodiments, the hydrogel exterior surface may be made to be bioactive. This bioactive surface, upon interaction with respective surfaces of spinous processes, will attach to the spinous process. The hydrogels with such a bioactive surface can then act as a drug delivery device/carrier in a manner known to those skilled in the art.

As mentioned above, casings suitable for implementing interspinous dynamic stabilization systems disclosed herein may take various forms and sizes. For example, some casings may attach to the adjacent spinous processes bi-laterally and some casings may attach to the adjacent spinous processes in the anterior-posterior direction. In some cases, existing inter-spinous devices may be utilized as casings to constrain and/or house the specially manufactured anisotropic hydrogels. Examples of suitable inter-spinous devices may include the coflex™ interspinous implant and the Wallis® System. The coflex™ interspinous implant, invented by Dr. Jacques Samani in 1994, can be obtained from Paradigm Spine, LLC of New York. An exemplary implementation is described below with reference to FIG. 9. The Wallis® System, developed by Abbott Spine, is an interspinous dynamic stabilization device intended to treat mild to moderate degenerative disc disease (DDD). It consists of a poly-ether-ether-ketone (PEEK) spacer and a pair of Dacron® retention bands. The spacer is placed between two adjoining spinal processes and is held into place by bands, which are passed around the spinal processes and tightened to secure the device. As a complete construct, the device limits motion, both in flexion (the bands) and extension (the spacer). Rotational motion is also restricted, further stabilizing the motion segment.

FIG. 9 depicts one embodiment of interspinous dynamic stabilization system 700 comprising casing 750 and hydrogel 720. In this example, casing 750 comprises pocket 752 for housing hydrogel 720. Pocket 752 may be partially enclosed or fully enclosed (not shown) and is structured to be inserted in the space between superior spinous process 203a and inferior spinous process 203b. In some embodiments, pocket 752 may have an upper surface that conforms to the bottom portion of superior spinous process 203a and a lower surface that conforms to the top portion of inferior spinous process 203b.

Casing 750 further comprises four tabs 751 for attaching casing 750 to superior spinous process 203a and inferior spinous process 203b. In this example, two tabs 751 extend upwardly from either end of pocket 752 and two tabs 751 extend downwardly from either end of pocket 752. Tabs 751 and pocket 752 are formed monolithically out of a biocompatible material. Each tab 751 may have at least one hole 760 through which bone fastener 780 can be fastened or otherwise secured onto a spinous process. Suitable bone fasteners 780 may include, but are limited to, bone screws. In the example of FIG. 9, in a surgical procedure, dehydrated hydrogel 720 is inserted through opening 753 into pocket 752 and hydrated to cause the eventual lifting of superior spinous process 203a. This lifting of superior spinous process 203a will minimize the painful articulation in facet joint 205a. Along with adding the dampening/spring effect via hydrogel 720, interspinous dynamic stabilization system 700 can also add stability to the weakened spinal segment as casing 750 is relatively rigid by virtue of its structure. Opening 753 may be modified to close or partially close pocket 752. Due to the fact that casing 750 is attached to both superior spinous process 203a and inferior spinous process 203b, interspinous dynamic stabilization system 700 can have enhanced stability against torsional and lateral bending. As described below with reference to FIG. 12, the casing itself can be made to have its own spring/dampening factor for enhanced ROM. Thus, in some embodiments, interspinous dynamic stabilization system 700 can be a viable solution to both facet joint pain and dynamic stabilization.

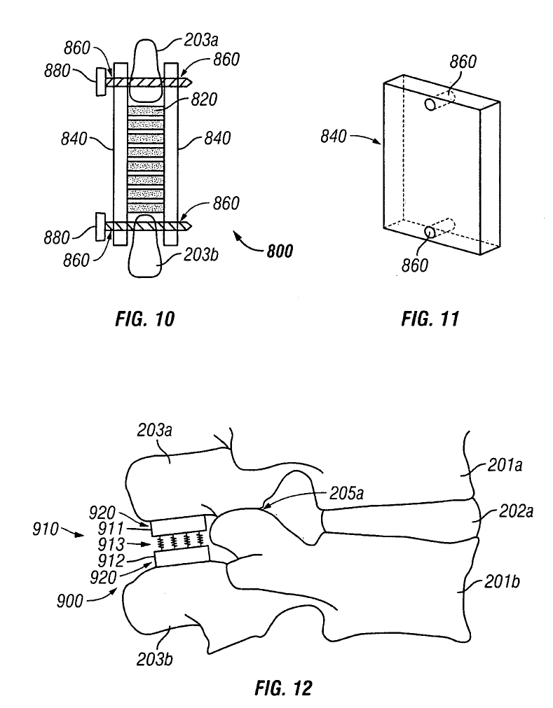

In some embodiments, casings of an interspinous dynamic stabilization system disclosed herein may be made without its top and bottom being enclosed. FIG. 10 depicts one embodiment of interspinous dynamic stabilization system 800 comprising casing 840 and hydrogel 820. In this example, casing 840 comprises two pieces of side walls, one of which is shown in FIG. 11. These walls can prevent hydrogel 820 from dispositioning. Each wall of casing 840 has two or more holes 860, with one being located close to the top of the wall for laterally attaching casing 840 to superior spinous process 203a and another one being located close to the bottom of the wall for laterally attaching casing 840 to inferior spinous process 203b, using bone fasteners 880. This embodiment can ensure that the topmost and bottom layer of hydrogel 820 would directly attach to the inferior surface of superior spinous process 203a and superior surface of inferior spinous process 203b, respectively. The exterior or interacting surface of hydrogel 820 can be made bioactive, making this embodiment of interspinous dynamic stabilization system 800 suitable for drug delivery applications.

FIG. 12 depicts one embodiment of interspinous dynamic stabilization system 900 comprising casing 910 and hydrogel 920. In this example, spring or dampening elements 913 are inserted between portion 911 and portion 912 of casing 910 for better ROM and dynamic stabilization. In some embodiments, each of portion 911 and portion 912 contains at least one unit of hydrogel 920. In some embodiment, a top surface of portion 911 can be adapted to conform to the bottom portion of superior spinous process 203a and a bottom surface of portion 912 can be adapted to accept dampening elements 913. Likewise, in some-embodiment, a top surface of portion 912 can be adapted to accept dampening elements 913 and a bottom surface of portion 912 can be adapted to conform to the top portion of inferior spinous process 203b.

Currently, there does not seem to be a dynamic stabilization system that utilizes a combination of an interspinous process implant and anisotropic hydrogels for spinal treatment. It is contemplated that embodiments of the interspinous dynamic stabilization system disclosed herein can be one of the most versatile systems in the market, providing solutions for dynamic stabilization and facet joint pain resulting from spinal instability and/or abnormal facet joint loading and articulation. Due to its attachment to spinous processes, the system would also offer enhanced stability for torsional and lateral bending ROM. Further, some embodiments of the system can be implemented to act as a drug delivery device/carrier. More importantly, embodiments of the interspinous dynamic stabilization system disclosed herein can be reversibly removed in case if the surgery is deemed unsuccessful.

Embodiments of an interspinous dynamic stabilization system have now been described in detail. Those skilled in the art will appreciate that any of the embodiments described above may be used individually or in combination with other spinal implants. Further modifications and alternative embodiments of various aspects of the disclosure will be apparent to those skilled in the art in view of this description. Accordingly, this description is to be construed as illustrative only and is for the purpose of teaching those skilled in the art the general manner of carrying out the disclosure. It is to be understood that the forms of the disclosure shown and described herein are to be taken as examples of embodiments. Elements and materials may be substituted for or implemented from those illustrated and described herein, as would be apparent to one skilled in the art after having the benefit of the disclosure. Changes may be made in the elements or to the features described herein without departing from the spirit and scope of the disclosure as set forth in the following claims and their legal equivalents.

What is claimed is:

1. An interspinous dynamic stabilization system, comprising:
at least one unit of hydrogel manufactured to expand axially in a predetermined direction upon absorption of fluid; and
a casing for constraining or housing said at least one unit of hydrogel, wherein said casing comprises a top surface conforming to a bottom portion of a superior spinous process and a bottom surface conforming to a top portion of an inferior spinous process and wherein said at least one unit of hydrogel in hydrated form lifts said superior spinous process.

2. The interspinous dynamic stabilization system of claim 1, wherein said casing has folds for accommodating expansion of said at least one unit of hydrogel.

3. The interspinous dynamic stabilization system of claim 1, wherein said casing is fully or partially enclosed.

4. The interspinous dynamic stabilization system of claim 1, wherein said casing comprises tabs for attaching to said superior spinous process and said inferior spinous process.

5. The interspinous dynamic stabilization system of claim 4, wherein said casing further comprises a pocket area where said at least one unit of hydrogel is to be constrained or housed.

6. The interspinous dynamic stabilization system of claim 5, wherein one of said tabs extends over said pocket area, leaving a gap through which said at least one unit of hydrogel is insertable.

7. The interspinous dynamic stabilization system of claim 1, wherein said casing comprises an upper portion, a lower portion, and dampening elements, wherein said top surface is part of said upper portion of said casing, wherein said bottom surface is part of said lower portion of said casing, and wherein said dampening elements are positioned between said upper portion and said lower portion of said casing.

8. The interspinous dynamic stabilization system of claim 1, wherein said casing is made of a metal or composite material.

9. An interspinous dynamic stabilization system, comprising:
at least one unit of hydrogel manufactured to expand axially in a predetermined direction upon absorption of fluid; and
a casing for constraining said at least one unit of hydrogel, wherein said casing comprises at least two side walls, wherein each of said at least two side walls has two or more holes, wherein each of said at least two side walls is laterally attachable to adjacent spinous processes using bone fasteners through said two or more holes, leaving a space between said adjacent spinous processes and said at least two side walls where said at least one unit of hydrogel is constrained and directly attachable to said adjacent spinous processes.

10. The interspinous dynamic stabilization system of claim 8, wherein an exterior or interacting surface of said at least one unit of hydrogel is bioactive.

11. The interspinous dynamic stabilization system of claim 8, wherein said casing is made of a metal or composite material.

12. A method of implanting an interspinous dynamic stabilization system, comprising:
making an incision in a patient;
placing a casing of said interspinous dynamic stabilization system between adjacent spinous processes of a spinal segment of the patient, wherein said adjacent spinous processes consist of a superior spinous process and an inferior spinous process, wherein said casing comprises a top surface conforming to a bottom portion of said superior spinous process, a bottom surface conforming to a top portion of said inferior spinous process, and an opening; and
inserting one or more units of a hydrogel in dehydrated form into said casing through said opening, wherein upon absorption of fluid said hydrogel expands axially in a predetermined direction, lifting said superior spinous process.

13. The method according to claim 12, further comprising:
supplying a saline solution to said hydrogel.

14. The method according to claim 12, further comprising:
preparing said bottom portion of said superior spinous process and said top portion of said inferior spinous process to accommodate said top surface and said bottom surface of said casing.

15. The method according to claim 12, wherein said hydrogel has a bioactive surface, further comprising:
utilizing said interspinous dynamic stabilization system as a drug delivery device.

16. A method of implanting an interspinous dynamic stabilization system, comprising:
making an incision in a patient;
attaching, bi-laterally or in an anterior-posterior direction, a casing of said interspinous dynamic stabilization system to adjacent spinous processes of a spinal segment of the patient, wherein said casing comprises an opening; and
inserting one or more units of a hydrogel in dehydrated form into said casing through said opening, wherein upon absorption of fluid said hydrogel expands axially in a predetermined direction, lifting said superior spinous process.

17. The method according to claim 16, further comprising:
supplying a saline solution to said hydrogel.

18. The method according to claim 16, wherein said adjacent spinous processes consist of a superior spinous process and an inferior spinous process and wherein said opening of said casing opens to a bottom portion of said superior spinous process and to a top portion of said inferior spinous process, further comprising:
determining whether said hydrogel in hydrated form directly touches both said bottom portion of said superior spinous process and said top portion of said inferior spinous process.

19. The method according to claim 16, further comprising:
repeating said inserting step until said hydrogel in hydrated form directly touches said adjacent spinous processes.

20. The method according to claim 19, wherein said hydrogel has a bioactive surface, further comprising:
utilizing said interspinous dynamic stabilization system as a drug delivery device.
